(19) United States
(12) Patent Application Publication
(10) Pub. No.: US 2026/0358229 A1
(43) Pub. Date: Nov. 12, 2026

(54) TORTUOSITY CALCULATION METHOD AND SYSTEM TAKING INTO ACCOUNT COMPLEX PORE-THROAT MORPHOLOGY AND STRUCTURE OF POROUS MEDIUM, AND MEDIUM AND PRODUCT

(71) Applicants: CHINA PETROLEUM & CHEMICAL CORPORATION, Beijing (CN); SINOPEC EXPLORATION & PRODUCTION RESEARCH INSTITUTE, Beijing (CN); HUAZHONG UNIVERSITY OF SCIENCE AND TECHNOLOGY, Hubei (CN)

(72) Inventors: Jun PU, Beijing (CN); Feifei GOU, Beijing (CN); Ting XU, Beijing (CN); Zhaoli GUO, Hubei (CN); Xuejie QIN, Beijing (CN); Wenchao FANG, Beijing (CN); Yue ZHANG, Hubei (CN)

(73) Assignees: CHINA PETROLEUM & CHEMICAL CORPORATION, Beijing (CN); SINOPEC EXPLORATION & PRODUCTION RESEARCH INSTITUTE, Beijing (CN); HUAZHONG UNIVERSITY OF SCIENCE AND TECHNOLOGY, Hubei (CN)

(21) Appl. No.: 19/719,215

(22) Filed: Jun. 25, 2026

Related U.S. Application Data
(63) Continuation of application No. PCT/CN2025/099488, filed on Jun. 6, 2025.

(30) Foreign Application Priority Data
Apr. 30, 2025 (CN) 202510560802.6

(51) Int. Cl. G01N 15/08; G01N 33/24

(52) U.S. Cl. CPC G01N 15/082; G01N 33/24

(57) ABSTRACT

The present disclosure belongs to the geological exploration field, and specifically discloses a tortuosity calculation method and system taking into account complex pore-throat morphology and structure of porous medium, and a medium and a product, which includes: dividing the pore-throat space image into several flow regions with streamlines as boundaries; for each of the flow regions, dividing the flow region into a plurality of targets connected in series along the streamline direction, with each target being equivalent to a regular rectangle; determining the tortuosity of the flow region based on the width of each rectangular target; and obtaining the tortuosity of the pore-throat space based on the tortuosity of each flow region.

### CROSS-REFERENCE TO RELATED APPLICATION

This application is a continuation of international application of PCT application serial no. PCT/CN2025/099488, filed on Jun. 6, 2025, which claims the priority benefit of China application serial no. 202510560802.6, filed on Apr. 30, 2025. The entirety of each of the above-mentioned patent applications is hereby incorporated by reference herein and made a part of this specification.

### BACKGROUND

### Field of the Disclosure

The present disclosure belongs to the geological exploration field, and more specifically, relates to a tortuosity calculation method and system taking into account a complex pore-throat morphology and a structure of a porous medium, and medium and a product.

### Description of Related Art

Tortuosity is a ratio of an actual path length traversed by a fluid particle within a porous medium to an apparent length, directly reflecting the degree of tortuousness and complexity of a porous medium. Tortuosity is an important parameter for the quantitative characterization of pore-throat structural features in hydrocarbon reservoirs. Accurate computation of tortuosity in porous media may provide fundamental parameters for reservoir quality characterization and lay the groundwork for numerical flow simulations, and is therefore of significant importance.

At present, the industry primarily employs three methods to obtain tortuosity: experimental methods, analytical methods, and numerical simulation methods. The experimental method entails determining macroscopic parameters of the porous medium, such as porosity and permeability, through laboratory testing, and then back-calculating tortuosity by means of empirical formulas. However, such formulas often contain empirical constants whose physical significance is not clearly defined, resulting in substantial randomness in the computational outcomes and making accuracy difficult to ensure. The analytical method consists of constructing simplified, idealized conceptual models of porous media and deriving analytical expressions for tortuosity pursuant to Poiseuille's equation or Darcy's law. However, the simplified models so constructed are severely distorted and fail to faithfully reflect the complexity of actual porous media. The numerical simulation method, based on digital imaging of cores, constructs realistic spatial models of porous media and computes tortuosity through numerical simulation or morphology-based theoretical frameworks, thereby improving computational accuracy. Nevertheless, this method remains constrained by current algorithms, which frequently approximate the pore-throat space as flow conduits of uniform diameter, thereby neglecting the influence of complex pore-throat geometries and structures.

In summary, existing methods for calculating tortuosity do not adequately capture the variability in the trajectories of fluid particles at different locations within real porous media, and consequently do not meet the requirements for high-precision computation and analysis.

### SUMMARY OF THE DISCLOSURE

In view of the above defects or needs of improvement in the related art, the present disclosure provides a tortuosity calculation method and system taking into account a complex pore-throat morphology and a structure of a porous medium, and medium and a product, with the purpose of improving the calculation accuracy of pore-throat space tortuosity to make it more conform to actual situation.

To achieve the above purpose, according to a first aspect of the present disclosure, a tortuosity calculation method taking into account a complex pore-throat morphology and a structure of a porous medium is provided, which includes the following steps:
Dividing a pore-throat space image into several flow regions by using streamlines as boundaries;
For each of the flow regions, dividing the flow regions into a plurality of targets connected in series along a streamline direction, with each target being equivalent to a regular rectangle; determining a tortuosity of the flow region based on a width of each of the rectangular targets;
Obtaining a tortuosity of the pore-throat space based on the tortuosity of each of the flow regions.

As a further preference, for any i-th flow region, the tortuosity of the flow region is determined based on the width of each of the rectangular targets, and the calculation formula is as follows:

$${τ}_{i} = \frac{\frac{{r}_{i , 1}^{3} + {r}_{i , 2}^{3} + … + {r}_{i , n}^{3}}{n}}{{( \frac{{r}_{i , 1} + {r}_{i , 2} + … + {r}_{i , n}}{n} )}^{3}}$$

Wherein, τᵢ represents a tortuosity of the i-th flow region; r_{i,j} represents the width of the j-th rectangular target in the i-th flow region; j=1, 2, . . . , n, and n represents a total number of targets of the i-th flow region.

As a further preference, the tortuosity of the pore-throat space is obtained based on the tortuosity of each of the flow regions, and the calculation formula is as follows:

$$\overline{τ} = \frac{{\sum }_{i = 1}^{m} {τ}_{i}}{m}$$

Wherein, τ̅ represents the tortuosity of the pore-throat space, τᵢ represents the tortuosity of the i-th flow region, and m represents a total number of the flow regions.

As a further preference, the pore-throat space image is obtained based on a two-dimensional image of a core.

As a further preference, the two-dimensional image of the core is a grayscale image, and the two-dimensional image of the core is converted into a binarized image of the core based on a preset grayscale threshold. The pore-throat space image is selected from a black part of the binarized image of the core.

As a further preference, the two-dimensional image of the core is obtained through cast thin section, electron microscope scanning, or CT.

As a further preference, the flow region is divided into the plurality of targets connected in series along the streamline direction. Specifically, flow boundaries on both sides of the flow region are equally divided into n parts, and the corresponding division points on the flow boundaries on the both sides are connected to form n targets connected in series.

According to a second aspect of the present disclosure, a tortuosity calculation system taking into account a complex pore-throat morphology and a structure of a porous medium is provided, which includes a processor. The processor is configured to execute the tortuosity calculation method taking into account a complex pore-throat morphology and a structure of a porous medium.

According to a third aspect of the present disclosure, a computer-readable storage medium is provided, in which a computer program is stored. When the computer program is executed by a processor, the tortuosity calculation method taking into account a complex pore-throat morphology and a structure of a porous medium is implemented.

According to a fourth aspect of the present disclosure, a computer program product is provided, which includes a computer program. When the computer program is executed by a processor, the tortuosity calculation method taking into account a complex pore-throat morphology and a structure of a porous medium is implemented.

In general, compared with the related art, the technical solution conceived by the present disclosure mainly has the following technical advantages:
The present disclosure, based on the theory of fluid mechanics, partitions the pore-throat space into a plurality of independent flow regions with streamlines serving as boundaries, and decomposes each flow region into a series of serially connected rectangular elements of regular morphology. On this basis, a tortuosity calculation method is established that accounts for the complex pore throat morphologies and structures of porous media, thereby enabling accurate computation of tortuosity within the pore-throat space and rendering the computational results more consistent with actual conditions. The method provides fundamental parameters for reservoir quality characterization and for studies of flow behavior in oil and gas reservoirs.

### BRIEF DESCRIPTION OF THE DRAWINGS

FIG. 1 is a two-dimensional digital image of a core according to an embodiment of the present disclosure.

FIG. 2 is a binarized image of a core according to an embodiment of the present disclosure.

FIG. 3 is a pore-throat space diagram intercepted according to an embodiment of the present disclosure.

FIG. 4 is a schematic diagram of flow region splitting according to an embodiment of the present disclosure.

FIG. 5 is a schematic diagram of equal division inside a flow region according to an embodiment of the present disclosure.

FIG. 6 is a schematic diagram of series connection of equivalent rectangles in a flow region according to an embodiment of the present disclosure.

### DESCRIPTION OF EMBODIMENTS

In order to make the objectives, technical solutions, and advantages of the present disclosure clearer, the present disclosure is further described in detail below with reference to the accompanying drawings and embodiments. It should be understood that the specific embodiments described herein are only used to explain the present disclosure and are not intended to limit the present disclosure. In addition, the technical features involved in the various embodiments of the present disclosure described below may be combined with each other as long as they do not conflict with each other.

A tortuosity calculation method taking into account a complex pore-throat morphology and a structure of a porous medium provided by an embodiment of the present disclosure includes the following steps:
(1) Acquiring a pore-throat space for which tortuosity needs to be calculated

Further, acquiring the pore-throat space based on a real digital image of a core porous medium includes:

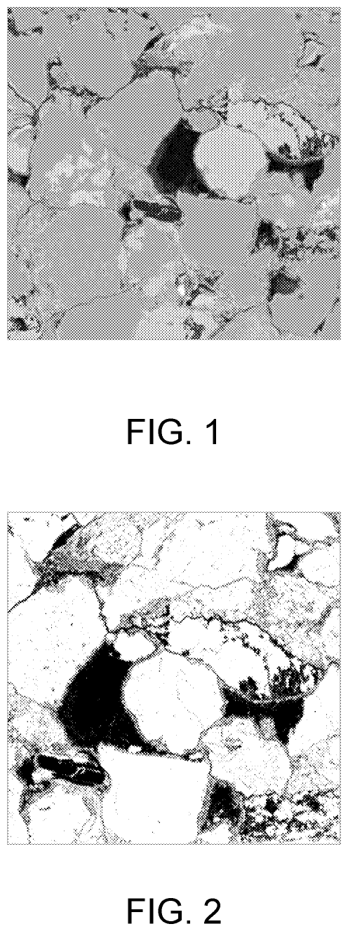

Acquiring a two-dimensional digital image of the core, as shown in FIG. 1, the image is a grayscale image, in which the light color represents rock mineral grains and the dark color represents the pore-throat space. The methods for acquiring the two-dimensional digital image of the core include but are not limited to cast thin section, electron microscope scanning, CT, and the like.

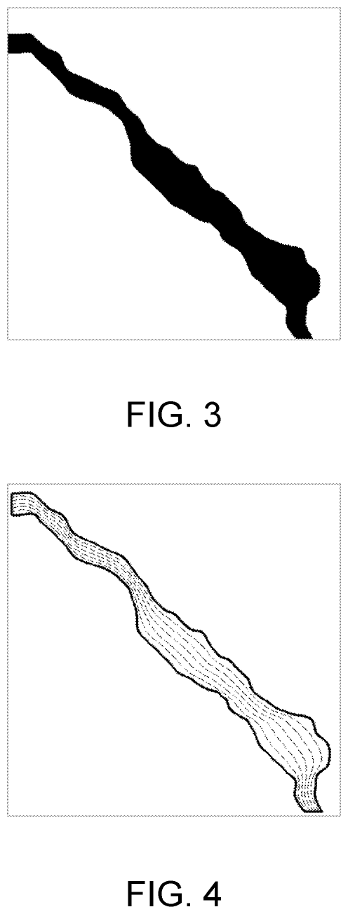

Selecting an appropriate grayscale threshold to obtain a binarized image of the core, as shown in FIG. 2, in which the black part represents the pore-throat space; selecting the pore-throat space for which tortuosity needs to be calculated therefrom, as shown in FIG. 3.
(2) Dividing the pore-throat space into m flow regions using streamlines as boundaries, as shown in FIG. 4

Specifically, the region division is performed based on a fluid motion trajectory. According to the basic theory of fluid mechanics, there is no fluid exchange between different flow regions, and each flow region may serve as an independent calculation unit. The flow regions may be equally or unequally divided, and for all cross-sections, a width ratio of the m flow regions remains consistent.
(3) Dividing each of the flow regions into a plurality of targets connected in series along a streamline direction

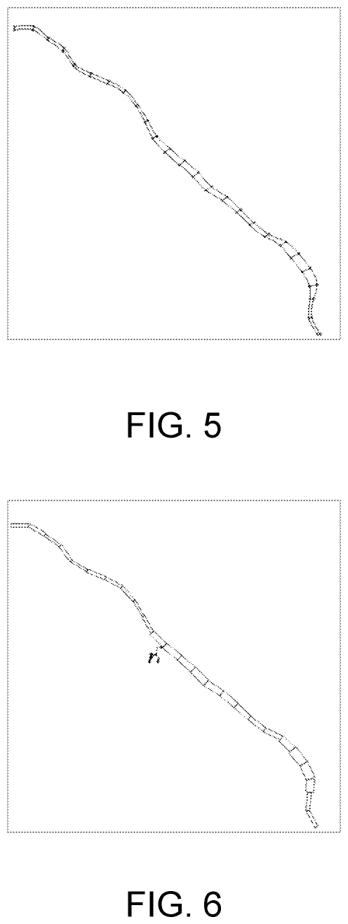

Specifically, for any i-th flow region, the boundary of the flow region is uniformly divided into n equal parts, and the corresponding division points on the flow boundary are connected to decompose the flow region into n targets connected in series, as shown in FIG. 5.
(4) When the number of decomposed targets is sufficiently large, based on the theory of calculus, each target may be regarded as a regular rectangle, as shown in FIG. 6, and the tortuosity of the flow region is determined according to a width of each of the rectangular target.

Specifically, the width r_{i,1}, r_{i,2}, . . . , f_{i,n} of each of the rectangular targets in the i-th flow region is obtained. Then a tortuosity τᵢ of the i-th flow region is calculated.

$${τ}_{i} = \frac{\frac{{r}_{i , 1}^{3} + {r}_{i , 2}^{3} + … + {r}_{i , n}^{3}}{n}}{{( \frac{{r}_{i , 1} + {r}_{i , 2} + … + {r}_{i , n}}{n} )}^{3}} \qquad ( 1 )$$

(5) By taking an arithmetic average of the tortuosity of all flow regions within the pore-throat space, the tortuosity τ̅ of such pore-throat space is determined as follows:

$$\overline{τ} = \frac{{\sum }_{i = 1}^{m} {τ}_{i}}{m} \qquad ( 2 )$$

Specifically, the derivation process is as follows:

Based on the basic theory of fluid mechanics, a calculation formula for a flow rate of a two-dimensional pore channel flow under a predetermined pressure difference is:

$$Δ p = \frac{3 q μ l}{2 {r}^{3}} \qquad ( 3 )$$

In the formula, Δp is a pressure difference between two ends of the channel; q is a channel flow rate; μ is a fluid viscosity; l is a distance between two ends of the channel; and r is a channel width.

The distance between two ends of the channel may be obtained as:

$$l = \frac{2 Δ {pr}^{3}}{3 q μ} \qquad ( 4 )$$

Based on the concept of calculus, formula (4) may be used to calculate an actual flow length of the i-th flow region as follows:

$${l}_{i} = {l}_{i , 1} + {l}_{i , 2} + … + {l}_{i , n} = \frac{2}{3 q μ} ( Δ {p}_{i , 1} {r}_{i , 1}^{3} + Δ {p}_{i , 2} {r}_{i , 2}^{3} + … + Δ {p}_{i , n} {r}_{i , n}^{3} ) \qquad ( 5 )$$

In the formula, lᵢ is the actual flow length of the i-th flow region; l_{i,n} is an actual flow length of the n-th rectangular target in the i-th flow region; Δp_{i,n} is a pressure difference between two ends of the n-th rectangular target in the i-th flow region; r_{i,n} is a width of the n-th rectangular target in the i-th flow region, and n is the number of rectangles in the flow region.

Because the flow region is divided into equal parts, and because the fluid flow velocity in the porous medium is extremely low, the influence of local hydraulic friction resistance may be neglected, and the following may be obtained:

$$Δ {p}_{i , 1} = Δ {p}_{i , 2} = … = Δ {p}_{i , n} = \frac{Δ {p}_{i}}{n} \qquad ( 6 )$$

By substituting Formula (6) into Formula (5), it follows that:

$${l}_{i} = \frac{2 Δ {p}_{i}}{3 q μ} \cdot \frac{{r}_{i , 1}^{3} + {r}_{i , 2}^{3} + … + {r}_{i , n}^{3}}{n} \qquad ( 7 )$$

In addition, according to the fluid mechanics method, an apparent length of the i-th flow region may be obtained as follows:

$${L}_{i} = \frac{2 Δ {p}_{i} {\overline{r}}_{i}^{3}}{3 q μ} = \frac{2 Δ {p}_{i}}{3 q μ} \cdot {( \frac{{r}_{i , 1} + {r}_{i , 2} + … + {r}_{i , n}}{n} )}^{3} \qquad ( 8 )$$

According to the calculation formula of tortuosity, the tortuosity of the i-th flow region may be obtained as follows:

$${τ}_{i} = \frac{{l}_{i}}{{L}_{i}} = \frac{\frac{{r}_{i , 1}^{3} + {r}_{i , 2}^{3} + … + {r}_{i , n}^{3}}{n}}{{( \frac{{r}_{i , 1} + {r}_{i , 2} + … + {r}_{i , n}}{n} )}^{3}} \qquad ( 9 )$$

By performing an arithmetic average of the tortuosity of different flow regions, the tortuosity of the target pore-throat space may be obtained.

An embodiment of the present disclosure further provides a tortuosity calculation system taking into account a complex pore-throat morphology and a structure of a porous medium, which includes a processor. The processor is configured to execute the tortuosity calculation method taking into account a complex pore-throat morphology and a structure of a porous medium.

An embodiment of the present disclosure further provides a computer-readable storage medium, in which a computer program is stored. When the computer program is executed by a processor, the tortuosity calculation method taking into account a complex pore-throat morphology and a structure of a porous medium is implemented.

An embodiment of the present disclosure further provides a computer program product, including a computer program. When the computer program is executed by a processor, the tortuosity calculation method taking into account a complex pore-throat morphology and a structure of a porous medium is implemented.

The following are specific embodiments:

Taking the pore-throat space shown in FIG. 3 as an example, the pore-throat space is divided into 6 flow regions with streamlines as boundaries, and each of the flow regions is equally divided into 25 small rectangles connected in series. Then the method of the present disclosure is adopted to calculate the tortuosity of the 6 flow regions and the tortuosity of the pore-throat space, and the calculation results are shown in Table 1.

TABLE 1
Calculation parameters and calculation results
of tortuosity of pore-throat space

| | Flow | Flow | Flow | Flow | Flow | Flow |
|---|---|---|---|---|---|---|
| | region 1 | region 2 | region 3 | region 4 | region 5 | region 6 |
| r₁ | 1.503 | 1.503 | 1.503 | 1.503 | 1.503 | 1.503 |
| r₂ | 1.694 | 1.424 | 1.667 | 1.18 | 1.424 | 1.424 |
| r₃ | 1.26 | 1.26 | 1.26 | 1.26 | 1.26 | 1.26 |
| r₄ | 1.471 | 1.471 | 1.471 | 1.471 | 1.471 | 1.471 |
| r₅ | 1.534 | 1.171 | 1.171 | 1.171 | 1.171 | 1.171 |
| r₆ | 1.513 | 1.542 | 1.542 | 1.542 | 1.542 | 1.542 |
| r₇ | 1.369 | 1.369 | 1.369 | 1.369 | 1.369 | 1.369 |
| r₈ | 1.618 | 1.25 | 1.25 | 1.25 | 1.25 | 1.25 |
| r₉ | 1.148 | 1.024 | 1.024 | 1.024 | 1.024 | 1.024 |
| r₁₀ | 1.248 | 1.37 | 1.37 | 1.37 | 1.37 | 1.37 |
| r₁₁ | 2.051 | 2.051 | 2.051 | 1.645 | 2.456 | 2.051 |
| r₁₂ | 2.903 | 2.903 | 2.903 | 2.903 | 2.903 | 2.903 |
| r₁₃ | 3.051 | 3.051 | 3.051 | 3.369 | 2.734 | 3.051 |
| r₁₄ | 3.193 | 3.193 | 3.193 | 3.193 | 3.193 | 3.193 |
| r₁₅ | 3.155 | 3.155 | 3.155 | 3.155 | 3.155 | 3.155 |
| r₁₆ | 3.097 | 3.097 | 3.097 | 3.097 | 3.097 | 3.097 |
| r₁₇ | 2.722 | 2.722 | 2.722 | 2.722 | 2.722 | 2.722 |
| r₁₈ | 2.557 | 2.557 | 2.557 | 2.557 | 2.557 | 2.557 |
| r₁₉ | 3.026 | 3.026 | 3.026 | 3.026 | 3.026 | 3.026 |
| r₂₀ | 3.491 | 3.491 | 3.491 | 3.491 | 3.491 | 3.491 |
| r₂₁ | 4.17 | 4.17 | 4.17 | 4.17 | 4.17 | 4.17 |
| r₂₂ | 4.085 | 4.085 | 4.085 | 4.085 | 4.085 | 4.085 |
| r₂₃ | 3.383 | 3.383 | 3.383 | 3.383 | 3.383 | 3.383 |
| r₂₄ | 1.668 | 1.668 | 1.668 | 1.668 | 1.668 | 1.668 |
| r₂₅ | 1.399 | 1.399 | 1.399 | 1.399 | 1.399 | 1.399 |
| τ | 1.513 | 1.572 | 1.558 | 1.614 | 1.559 | 1.572 |

| | |
|---|---|
| τ̅ | 1.564 |

Existing methods for calculating tortuosity fail to capture the variability in the trajectories of fluid particles at different locations within real porous media and are therefore inadequate to meet the requirements of high-precision computation and analysis. The present disclosure, starting from high-resolution digital core images and grounded in fundamental fluid mechanics, uses streamlines as boundaries to partition the pore-throat space into a plurality of independent flow regions, and decomposes each of the flow regions into a plurality of regularly shaped, serially connected rectangles, thereby establishing a tortuosity calculation method that accounts for the complex morphology and a structure of pore throats in a porous medium. The resulting calculations accord more closely with actual conditions and may provide fundamental parameters for reservoir-quality characterization and studies of flow behavior in oil and gas reservoirs.

Those skilled in the art will readily understand that the above descriptions are merely preferred embodiments of the present disclosure and are not intended to limit the present disclosure. Any modifications, equivalent substitutions, and improvements made within the spirit and principles of the present disclosure shall all be included within the protection scope of the present disclosure.

What is claimed is:

1. A tortuosity calculation method taking into account a complex pore-throat morphology and a structure of a porous medium, comprising the following steps:
dividing a pore-throat space image into several flow regions by using streamlines as boundaries;
for each of the flow regions, dividing the flow regions into a plurality of targets connected in series along a streamline direction, with each target being equivalent to a regular rectangle;
determining a tortuosity of the flow region based on a width of each of the rectangular targets;
obtaining a tortuosity of the pore-throat space based on the tortuosity of each of the flow regions.

2. The tortuosity calculation method taking into account the complex pore-throat morphology and the structure of the porous medium according to claim 1, wherein for any i-th flow region, the tortuosity of the flow region is determined based on the width of each of the rectangular targets, and the calculation formula is as follows:
$${τ}_{i} = \frac{\frac{{r}_{i , 1}^{3} + {r}_{i , 2}^{3} + … + {r}_{i , n}^{3}}{n}}{{( \frac{{r}_{i , 1} + {r}_{i , 2} + … + {r}_{i , n}}{n} )}^{3}}$$
wherein, τᵢ represents a tortuosity of the i-th flow region, r_{i,j} represents a width of a j-th rectangular target in the i-th flow region, j=1, 2, . . . , n, and n represents a total number of targets of the i-th flow region.

3. The tortuosity calculation method taking into account the complex pore-throat morphology and the structure of the porous medium according to claim 1, wherein the tortuosity of the pore-throat space is obtained based on the tortuosity of each of the flow regions, and the calculation formula is as follows:
$$\overline{τ} = \frac{{\sum }_{i = 1}^{m} {τ}_{i}}{m}$$
wherein, τ̅ represents the tortuosity of the pore-throat space, τᵢ represents a tortuosity of an i-th flow region, and m represents a total number of the flow regions.

4. The tortuosity calculation method taking into account the complex pore-throat morphology and the structure of the porous medium according to claim 1, wherein the pore-throat space image is obtained based on a two-dimensional image of a core.

5. The tortuosity calculation method taking into account the complex pore-throat morphology and the structure of the porous medium according to claim 4, wherein the two-dimensional image of the core is a grayscale image, and the two-dimensional image of the core is converted into a binarized image of the core based on a preset grayscale threshold;
the pore-throat space image is selected from a black part of the binarized image of the core.

6. The tortuosity calculation method taking into account the complex pore-throat morphology and the structure of the porous medium according to claim 4, wherein the two-dimensional image of the core is obtained through cast thin section, electron microscope scanning, or CT.

7. The tortuosity calculation method taking into account the complex pore-throat morphology and the structure of the porous medium according to claim 1, wherein the flow region is divided into the plurality of targets connected in series along the streamline direction, specifically, flow boundaries on both sides of the flow region are equally divided into n parts, and corresponding division points on the flow boundaries on the both sides are connected to form n targets connected in series.

8. A tortuosity calculation system taking into account the complex pore-throat morphology and the structure of the porous medium, comprising a processor, wherein the processor is configured to execute the tortuosity calculation method taking into account the complex pore-throat morphology and the structure of the porous medium according to claim 1.

9. A computer-readable storage medium, in which a computer program is stored, wherein when the computer program is executed by a processor, the tortuosity calculation method taking into account the complex pore-throat morphology and the structure of the porous medium according to claim 1 is implemented.

10. A computer program product, comprising a computer program, wherein when the computer program is executed by a processor, the tortuosity calculation method taking into account the complex pore-throat morphology and the structure of the porous medium according to claim 1 is implemented.
